(19) United States
(12) Patent Application Publication
(10) Pub. No.: US 2009/0246823 A1
(43) Pub. Date: Oct. 1, 2009

(54) EVALUATION METHODOLOGY OF THE PROTECTION CHARACTERISTICS OF PERSONAL PROTECTIVE EQUIPMENTS AGAINST BIOLOGICAL AGENTS

(75) Inventor: Stefano Cerbini, Pesaro (IT)

Correspondence Address: YOUNG & THOMPSON, 209 Madison Street, Suite 500, ALEXANDRIA, VA, 22314, US

(73) Assignee: CL.COM S.R.L., Forlì (IT)

(21) Appl. No.: 12/297,109

(22) PCT Filed: Apr. 3, 2007
(86) PCT No.: PCT/IT2007/000250

§ 371 Date: Oct. 14, 2008

(30) Foreign Application Priority Data
Apr. 12, 2006 (IT) PS2006A000008

(51) Int. Cl. C12Q 1/06; C12Q 1/02; C12M 1/34

(52) U.S. Cl. 435/39; 435/29; 435/287.1

(57) ABSTRACT

New testing methodology for the evaluation of the protection of the Personal Protective Equipments (PPE) for the respiratory tract against biological agents, characterized in that different machineries are used to reproduce the usage of the PPE, simulating a breathing through the Sheffield's head and self-respirator. The apparatus consists of: a) a viral and/or bacterial aerosol generator b) a test chamber containing the Sheffield's head c) a respirator simulating breathing and adjusting the inspiration and expiration frequency d) a suction system delivering the samples of air withdrawn in different points to the bubblers to determine the viral and/or bacterial concentrations.

### FIELD OF THE INVENTION

The present invention relates to testing methodology for the evaluation of the protection against biological agents of protective equipments for the respiratory tract characterized in that different machineries are used to reproduce the real usage of the protective equipment

### BACKGROUND ART

There are many testing methodologies to evaluate the efficacy of the respiratory tract protective equipments, and in particular to calculate the inward loss of sealing (European Standard EN 13274-1) and the respiratory resistance (European Standard EN 13274-3).

As everybody knows, there are also many methodologies to calculate the viral removal efficiency of the filtering materials used as filtering membranes in laboratories, in the pharmaceutical industry or in devices for medical purposes. These methodologies use innocuous aerosol challenge and bacteriophage.

An example of such methodologies is quoted in “Efficacy of a pleated hydrophobic filter as a barrier to Mycobacterium Tuberculosis transmission within breathing systems” (S. Speigh et al.—Centre for Applied Microbiology & Research—Porton Down, Salisbury, Wiltshire SP4 0JG, UK).

However all these methodologies have never been applied to respiratory tract protective equipments; in fact till now it hasn't been possible to test the bacteria and viral removal efficacy directly on the personal protective equipment while simulating breathing and the real application on the user's face.

In order to calculate the protective performance of the personal protective equipment (as filters for full face masks, half-face masks, filtering face masks, etc. . . . ) against biological agents as bacteria and viruses, the analytical methodologies using dusts and/or different kinds of chemical aerosols made up of non-vital substrata, have always been used.

However, all these testing methodologies are representative neither of the behaviour of the microbial agent nor of its properties of penetrating the barrier media which is a protective equipment for the respiratory tract.

### DESCRIPTION OF THE INVENTION

The present invention refers to a new testing methodology for the evaluation of the protection of the Personal Protective Equipments (PPE) for the respiratory tract against biological agents, characterized in that different machineries are used to reproduce the usage of the PPE, simulating a breathing through the Sheffield's head and self-respirator.

A particular embodiment of the present invention is the whole apparatus used to evaluate the protective skill of the PPE against biological agents as well as the use of the Sheffield's head and of the self-respirator to evaluate the protective skill of the PPE against biological agents.

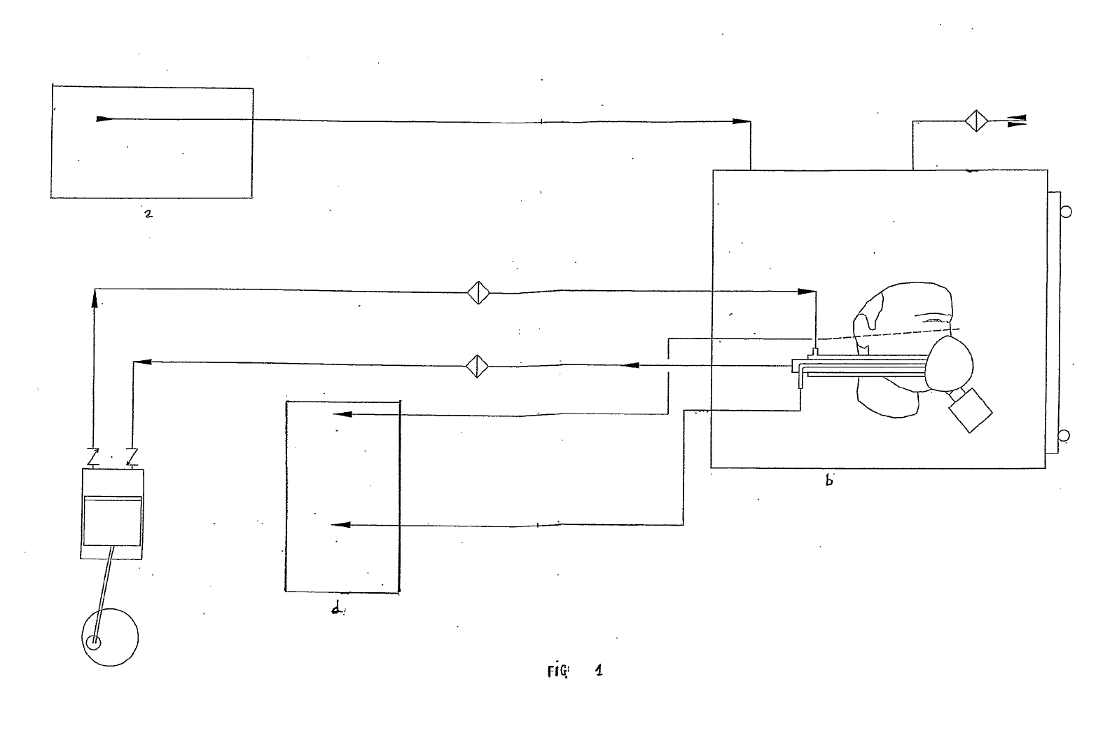

FIG. 1 outlines a scheme of the apparatus used.

The apparatus consists of:

a) a viral and/or bacterial aerosol generator
b) a test chamber containing the Sheffield's head
c) a respirator simulating breathing and adjusting the inspiration and expiration frequency
d) a suction system delivering the samples of air withdrawn in different points to the bubblers to determine the viral and/or bacterial concentrations.

This methodology mainly consists in generating an aerosol of a test micro-organism by means of the generator (a) and in sending the aerosol to the test chamber (b).

Inside the test chamber there is the Sheffield's head on which the PPE is placed.

The air inside the test chamber is inhaled by the Sheffield's head through the self-respirator (c).

Modification on the Sheffield's head are done in order to allow the inhalation of the air through the mouth-nose area and the exhalation from the back side of the head to be sent to the self-respirator.

Thanks to the placement of the PPE directly on the Sheffield's head, during the test implementation to check the protective efficacy of the PPE, the surround properties linked to the physical/mechanical and ergonomic characteristics of the PPE (which are essential for the PPE to be suitable to the protection uses against biological agents) are also taken into consideration.

That is to say that possible passages of contaminants due to the loss of sealing owing to a bad wearing of the PPE (and therefore not depending on the filtering properties of the filtering material itself) are also evaluated.

In order to calculate the viral or bacterial retention of the PPE, two testing analyses are performed on the air withdrawn through the suction system (d) and sent to some bubblers.

In particular the microbial concentrations in the air inside the test chamber (white test, withdrawn from position correspondent to the right eye of the Sheffield's head), and in the air which passed through the PPE (sample test, withdrawn below the PPE, correspondent to the mouth) are calculated.

Both the white test and the sample test are carried out simultaneously through bubbling, for a specific period of time, in a solution having both the appropriate composition and the right acidity/alkalinity (pH), through two separate tubes, the dispersion of biological agents in the test chamber (white test) and the sample of air crossing the PPE (sample test).

The two bubblers are both connected to a suction system at a constant flow.

At the end of the testing, the solutions contained into the bubblers are moved into appropriate sterile containers and then the collected micro-organisms are counted.

The ratio of micro-organisms blocked by the PPE is determined as follows:

$$\% ( blocked micro \text{-} organism rate ) = 100 - ( \frac{Na}{Nv} X 100 )$$

wherein:
Nv=concentration of the test micro-organism in the aerosol inside the test chamber (white test)
Na=concentration of the test micro-organism below the filtering face masks (sample test)

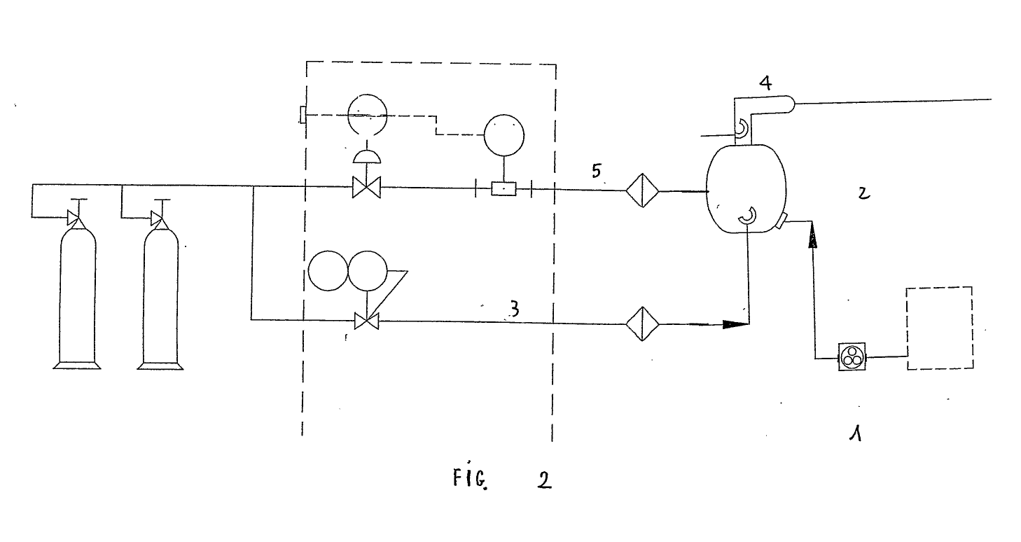

FIGS. 2-6 show in details the processing flow and its components

The aerosol generator (FIG. 2) is made up of:
peristaltic pump (1)
nebuliser (2)
nebulisation line (3)
desiccation tube (4)
flow tube (5)

A suspension of a known quantity of a micro-organism is fed up, through a peristaltic pump (1), into the nebuliser (2) where the compressed air, passing through the nebulisation line (3), creates an aerosol.

This aerosol, once fed into the desiccation tube (4), is mixed with the dry compressed air which comes separately from the flow line (5).

The microbial aerosol droplets entering the desiccation tube, evaporate quickly and are moved to the test chamber with a constant flow.

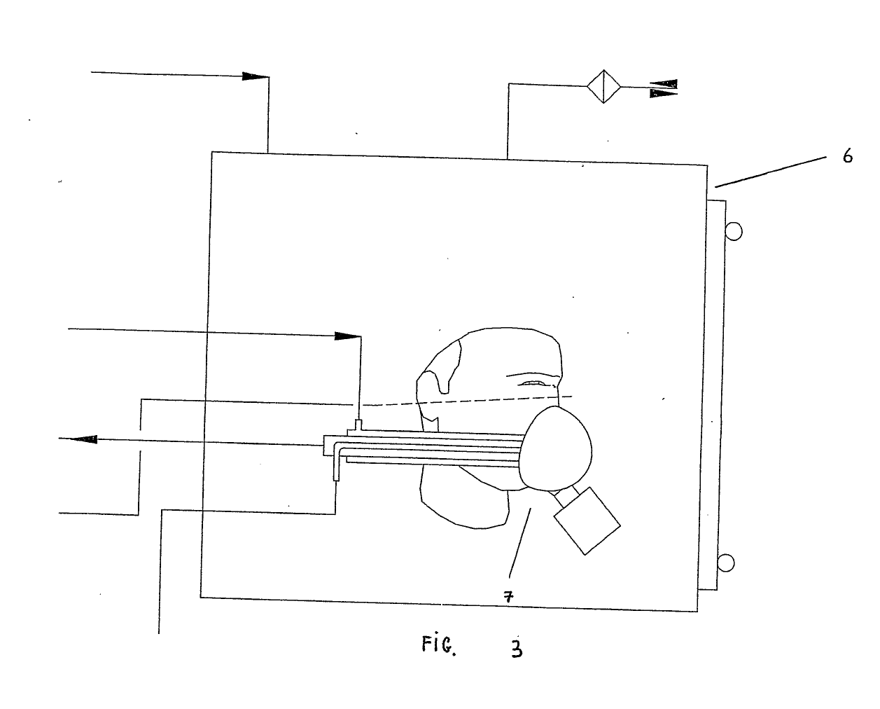

The test chamber (FIG. 3) is mainly set up of:
a hermetically sealed container (6)
one Sheffield head (7), put into the container, with piping lines.

The shape and dimensions of the container (6) allow a Sheffield head to be installed and the container is made of a material able to guarantee the air tight; to this purpose, the walls are sealed, with gaskets, and one of them can be opened to allow movements required by the methodology.

The Sheffield head (7) is put into the hermetic container (6) and it is complete with connecting piping to the self-respirator and with piping for the withdrawing of air samples contained into the test chamber and of the air passing through the PPE.

The hermetic container is shaped as a parallelepiped conforming to the size of a lab table, with an folding opening/closing wall and it is typically made of Lexan or similar materials.

The Sheffield head is for example complete with an accessory having three concentric tubes, two of which are linked to the automatic respirator and the third one collects the air passed through the PPE at the mouth nose level of the head; one more tube, the fourth one, is placed at the right eye level and withdraws the air contained into the test chamber.

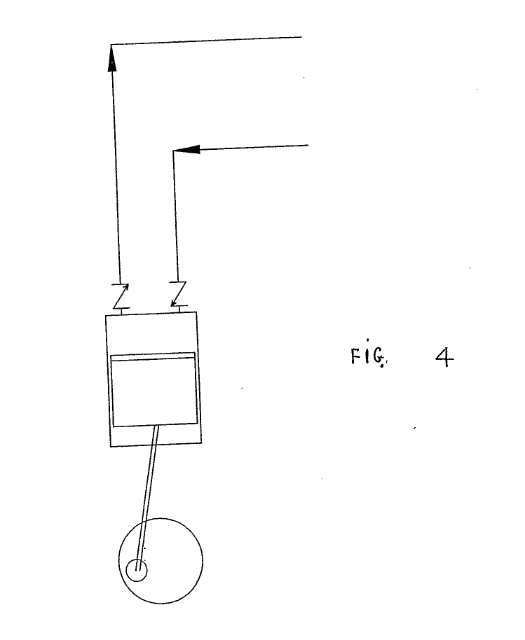

The respirator (FIG. 4) is made up of a pump and an inverter which regulates the inhalation/exhalation speed; the respiration frequency can be adjusted as per the normal human respiration and is typically comprised between 20 and 40 cycles per minute with an air volume ranging between 1.5 and 3.5 litres per cycle.

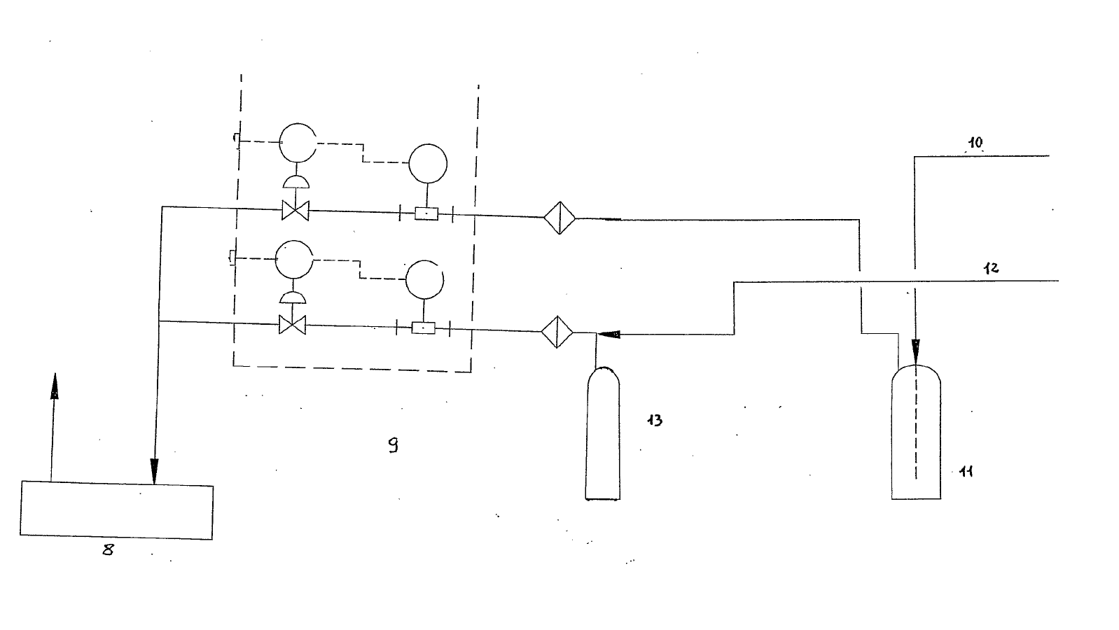

The suction system (FIG. 5) is mainly made up of:
vacuum pump (8)
flow regulator (9)
white test inhalation tube (10)
white test bubbler (11)
sample test inhalation tube (12)
sample test bubbler (13)

The system sucks the dispersion of the micro-organisms into the test chamber to quantify them.

The suction occurs through a vacuum pump (8) that allows the withdrawal at a constant flow which is adjusted and controlled through the flow regulators (9).

Both the white test and the sample test are carried out simultaneously, through two different lines, by bubbling the micro-organism dispersions in an appropriate and pH controlled solution. The dispersion used to collect viral agents is typically a pH 6.8 solution, while for bacterial agents, the pH is typically neutral.

The dispersion in the test chamber (white test) is withdrawn at the right eye level of the Sheffield head through the inhalation tube (10) and it bubbles into the sterile glass bubbler (11).

The air sample passing through the PPE (sample test) is withdrawn at the Sheffield head mouth level through the inhalation tube (12) and it bubbles into the sterile glass bubbler (13).

At the end of the test, the bubblers are disconnected, the solutions are delivered into sterile containers and the count of the micro-organisms in the solutions is done.

A particular embodiment of the present invention is the apparatus used to check the protective efficacy of the PPE against biological agents characterized in a viral and/or bacterial aerosol generator, a test chamber containing the Sheffield's head, a respirator simulating breathing and adjusting the inspiration and expiration frequency, a suction system delivering the samples of air withdrawn in different points to the bubblers to determine the viral and/or bacterial concentrations, the Sheffield head being equipped with pipes connected to the self-respirator, to allow the inhalation and the exhalation of the air through the mouth-nose area, and with drawal pipes of both the air contained into the test chamber and the air passing through the PPE.

A preferred embodiment of the present invention is the apparatus evaluating the protective efficacy against biological agents of the PPE, where the Sheffield head is placed into the test chamber in Lexan, is equipped with an accessory having three concentric tubes, two of which are linked to the automatic respirator and the third one collects the air passed through the PPE at the mouth-nose level of the head, and with one more tube, the fourth one, placed at the right eye level and withdrawing the air contained into the test chamber, and where the respirator is made up of a piston pump and of an inverter which regulates the inhalation/exhalation speed.

Further particular embodiments of the present invention are also the use of the Sheffield head modified as above described and of the self-respirator made up through a pump to evaluate the PPE protective efficacy against biological agents.

This methodology can be used to determine the protection against biological agents, and in particular against bacteria and viruses.

The preparation of the challenge suspensions and the count of the micro-organisms can be carried out by any process known for these use; the processes are typically different for viruses and bacteria

In order to explain this invention at best, here follows two examples of methodologies used respectively for viral and bacterial agents.

### EXAMPLE 1

### Viral Agents Methodology

In the example here described the testing micro-organism used is the bacteriophage MS-2 (National Collection of Industrial Bacteria: NCIMB 10108) which is a polyhedric vilus sized 0.02 μm. The number of active MS-2 bacteriophage in the challenge suspension and stored after the processing, is determined setting up a dispersion methodology on agar layer.

The count methodology consists on withdrawing aliquots ranging from 0.1 ml. or 1 ml of pH 6.8 “Buffer Phage” containing MS-2 bacteriophage and on mixing them, in one case, with 2.5 ml Tryptone soya agar containing about 0.5 ml of stationary growth (4-6 hours from inoculating) of Escherichia Coli NCIMB 9481 (approx. 10⁸ CFU/ml), in the second case, with 5 ml of Tryptone soya agar containing about 1.0 ml of stationary growth (4-6 hours from inoculating) Escherichia Coli NCIMB 9481 (approx. 10⁸ CFU/ml).

The agarized soft bed is thus immediately poured onto Tryptone soya agar plates in order to make a double layer. After 24-hour incubation at 37° C., the bacteriophage visible plaques were counted. The plates showing visible lysis plaques (pfu: plaque forming units) are selected and multiplied by the respective dilution. Pfu so determined will be equivalent to the number of MS-2 bacteriophages in the Buffer solution.

Suspensions at a known titre of challenge are firstly prepared, by diluting the original suspension in a Buffer Phage. Dilutions are then prepared and, through the “Double layer” method, the concentration is checked. Once the count finishes, the plates of the highest dilution which show a confluent lysis must be selected. A rate of Buffer Phage is added to these plates and, using a sterile spatula, the agar is broken and mixed with the Buffer.

In a sterile container, the Buffer containing agar is stored and decanted, then it is shaken vigorously until the agar breaks. The results is spun; the agar residuals will constitute the deposit. The floating is taken and filtered with a membrane and the aliquots are stored at 4° C. A, so prepared, suspension at a known titre is inserted into the aerosol generator with a definite volume.

The viral suspension is driven to the nebuliser (2) through the flow generated by the pump (1).

The aerosol generator works then both through the pressure of the aerosol spray (3) and the desiccation flow of the line (5)

After waiting for the test chamber homogeneous filling up, once the chamber is filled in, the respirator, and the vacuum pump of the suction system are activated. Both white and sample tests are carried out simultaneously through two separate tubes while bubbling in the Buffer phage the dispersion withdrawn. At the end of the test, both bubblers are disconnected, the solutions are transferred into appropriate sterile containers and the containers are stored immediately at 4° C. in order to inhibit any microbial growth.

The alive MS-2 Bacteriophages collected through the bubbling sampler are calculated using the double layer methodology described above.

### EXAMPLE 2

### Bacteria Agents Methodology

The protection efficacy against bacteria agents was determined through a similar procedure to that of viral agents, but the test micro-organism was collected through bubbling in a pH 7.0 diluent. The micro-organism used for testing is the Brevundimonas diminuta (ATCC 19146), a bacterium sized 0.3 μm. The bacterial suspension was prepared as follows: some under-cultures are prepared from a stock culture, through a creep on Tryptone Soia Agar plate and stored at 30-35° C. for 18-24 hours. After that, a further under-culture is prepared to be taken from the first one, in the same way, and is stored at 30-35° C. for 18-24 hours. The second under-culture is the working culture.

The working culture is withdrawn and put into a pH 7.0 diluent in a beute.

The beute is stirred with a mechanic stirrer and, then, the suspension is taken and put into a test-tube.

The number of cellules in the suspension must reach a value between 1×10⁷ CFU/ml and 1×10¹⁰ CFU/ml, using the dilution and estimating the quantity of units, through the McFarland index.

A bacterial suspension count is then performed.

The suspension must be stored into the fridge at 2-8° C. to be used during the day.

The count of the bacterial suspensions, tested and collected downstream, was performed as follows:

serial dilutions of the suspension to be counted are prepared using the diluent. A twin sample of each dilution is mixed and taken and the sample is carried in Petri plates. A determined quantity of TSA as a liquid is added, kept in bain-marie at 45° C., shaking the plate gently. The plates are incubated at 30-35° C. for 24 h. After the count of the number of units for each plate, the plates are incubated for further 24 hrs. The number of units grown on each plate are counted again, without counting the ones not well separated.

The highest number of units for each sample is determined.

The number of CFU/ml of the test suspension is calculated.

The above described examples of methodologies have the only aim of explaining the invention better and they do not imply any limitations.

For example, micro-organisms having similar sizes and microbiological characteristics can be used as well, and also alternative test suspension preparations, different times and collecting methodologies and different counting ways.

The methodology can be used to evaluate the efficacy against biological agents of all the PPE for the respiratory tract as, for example, full face masks, filtering face masks, disposable cup-shaped or fold-flat filtering face masks, filters.

For example the processing in example 1 has been used to calculate the efficacy of a fold-flat filtering face mask like the one described in the WO 2005/077214 A1 patent characterized in a filtering layer composed of borosilicate micro-glass fibers bound together by a vinyl acetate resin, the fiber matrix being supported by a strong, cellulose based, substrate and the structure being treated with a silicone based coating.

The results are the following:

| | Virus inside the | Virus found after | |
|---|---|---|---|
| virus into | test chamber | the the filtering face | Blocked virus % |
| challenge | White test (Nv) | mask Sample Test (Na) | 100 − |
| (×10⁹) | (×10⁵) | (×10³) | (Na/Nv × 100) |
| 4.6 | 3.4 | 1.7 | 99.5000 |
| 4.6 | 7.5 | 1.3 | 99.8267 |
| 4.6 | 4.9 | 1.0 | 99.7959 |
| 3.1 | 4.5 | 1.2 | 99.7333 |
| 3.1 | 6.4 | 2.1 | 99.6719 |
| 3.1 | 4.6 | 1.4 | 99.6957 |

The methodology object of the present invention has been validated in compliance with the Good Laboratory Practice (GLP) headlines.

### Validation of the Methodology

Validation testing of the methodology took into consideration both the test system efficiency (micro-organisms suspension uniformity of distribution into the buffers, viability of the micro-organism during the test implementation, whole method precision) and the analytical efficiency of the microbial agents counting (repeatability, intermediate precision, accuracy).

The validation testing have been carried out concerning both the viral agents method using the MS-2 bacteriophage, and the bacteria agents method using the Brevundimonas diminuta.

The efficiency of the micro-organism suspension counting has been checked first.

The concentration of a micro-organisms suspension was calculated by the appropriate counting methodology. For each dilution the titre has been checked many times and the test repeated several times.

The results have been used to calculate the repeatability of the analytical results, that is to say the precision, checking repeatedly in a homogeneous sample the suspension count.

For each dilution chosen, average and standard deviation were counted. Then, the Variation Rate Percentage (CV %) was calculated, obtained from a percentage rate between the standard deviation and the average of the measurements done.

CV %=sigma/Y*100

where:
sigma=standard deviation
Y=values average

The CV value obtained for the different dilutions was lower than 15% both in the virus and in the bacteria performances, showing a right repeatability of the count.

Using the results obtained in the above described six testing, the methodology accuracy has been checked, that is to say, how the experimental value differs from the theoretic known data.

The criteria of evaluation is based on the recovery %, which is the rate of the experimental data and the theoretic known data.

Recovery %=Ns/N*100

where:
N=viral suspension theoretic known data (pfu/ml)
Ns=viral suspension experimental data obtained (pfu/ml)

The recovery values % obtained in the 12 tests turned out to be all included in the range 70-130%, both in case of viruses and of bacteria.

The average value of recovery %, both in the tests carried out using viruses and in the ones using bacteria, ranged between 80-120%

Finally, the tests to calculate the repeatability were carried out by two different operators in two different days. Therefore, the intermediate precision of the analytical results was calculated as well, to be taken as the precision depending on the days and on the different people.

The CV value obtained for the different dilutions by the two operators in two different days, was less than 15% both in the case of viruses and in the case of bacteria.

This result indicates a good intermediate precision.

Also the test system efficiency was checked.

A work culture was prepared as already showed in the method description and it was fed the aerosol device with a definite volume of micro-organisms suspension. The aerosol generator was turned on and waited for the time to allow an homogeneous fill up of the test chamber. Then, the vacuum pump of the suction system was turned on and then both the vacuum pump and the aerosol generator were turned off.

Both bubblers were disconnected from the circuit and the solutions were moved into sterile containers kept at 4° C. in order to inhibit any microbial growth.

Some counting tests were then performed with the required dilutions on the buffer solutions using the method suitable to the kind of micro-organism.

The test was performed many times in two different working days.

The distribution uniformity of the micro-organism suspension into the two bubblers (white test and sample test) was calculated as well as in the two withdrawal points inside the chamber.

For each test, the difference between the percentage of the micro-organisms collected in the two bubblers was calculated, using the following formula:

$$Percentage of distribution difference ( R \% ) = \frac{Na}{Nv} * 100$$

where:
Nv=micro-organisms count performed into bubbler 11 (pfu/ml)
Na=micro-organisms count performed into bubbler 13 (pfu/ml)

No distribution difference value was out of the range ±5% both in the case of viruses and in the bacteria one. Therefore, the micro-organisms distribution in the different points turned out to be suitable.

The whole methodology precision has then been evaluated in terms of repeatability of the analytical results.

Using the data outcome of these previous tests for both the bubblers, the CV % was calculated through the following formula:

CV %=sigma/Y*100

where:
sigma=standard deviation
Y=average value for n samples

The CV % obtained in the two days was >25%, both in the case of viruses and in the one of bacteria, which indicates a suitable repeatability of the analytical results and therefore of a suitable whole methodology precision.

Finally, the viability of the micro-organism during the test implementation was determined, that is to say, the ability of the micro-organism to be viable for at least 30 minutes, allowing the test chamber to keep a sufficient concentration in order to outline, under the analytical aspect, the protection efficiency of the PPE.

A count of micro-organisms suspension, having a known titre, between 1×10⁷ and 1×10⁸, was performed immediately after its preparation (To).

The count of the same suspension was performed after 15, 30 and 45 minutes after the preparation (T₁₅, T₃₀, T₄₅).

The T₁₅, T₃₀ and T₄₅ count obtained was then compared to To.

The test was repeated three times.

The titre decrease at T15, T30 and T45 was less than 2 logarithms in comparison with the viral titre at To, both in the case of viruses and in the one of bacteria.

Therefore, the micro-organisms are always viable during the testing.

Although particular embodiments of the present invention have been described in the foregoing description, it will be understood by those skilled in the art that any simple modification and rearrangement will not depart from the spirit or essential attributes of the invention which are defined in the following claims.

1. Testing methodology for the evaluation of the protection of the Personal Protective Equipments (PPE) for the respiratory tract against biological agents, characterized in that the usage of the PPE is reproduced, by simulating a real breathing through the Sheffield's head and a self respirator; thanks to the placement of the PPE directly on the Sheffield's head and to the breathing simulation, besides the efficacy of the PPE, the surround properties linked to the physical/mechanical and ergonomic characteristics of the PPE are taken into consideration which are essential for the PPE to be suitable to protect against biological agents, that is to say that possible passages of contaminants due to the loss of sealing/leakages or to defects, and therefore not depending on the filtering properties of the filtering material itself, are also taken into consideration.

2. Methodology as claimed in claim 1, characterized in generating a test micro-organism aerosol through a viral or bacterial aerosol generator and in moving of the aerosol to a test chamber inside of which there is a Sheffield's head on which there is placed a PPE, the test chamber being connected to a respiratory device and to a suction system for sampling.
The air inside the test chamber is inhaled by the Sheffield's head, through the respirator that simulates actual breathing, by adjusting the inhalation and exhalation frequency and the Sheffield's head is modified in order to allow the inhalation of the air through the mouth-nose area and the exhalation from the back side of the head to be sent to the self-respirator.
The suction system sends samples of air withdrawn in different points to some bubblers in order to calculate the microbial concentrations in the air inside the test chamber (white test), and in the air which passed through the PPE (sample test).
Both the white test and the sample test are carried out simultaneously by bubbling, for a specific period of time and at a constant flow, in a solution having both a suitable composition and a suitable pH, through two separate tubes, the dispersion of biological agents in the test chamber and the sample of air which crosses the PPE.
At the end of the testing, the solutions contained into the bubblers are moved into appropriate sterile containers and then the collected micro-organisms are counted.
The ratio of micro-organisms blocked by the PPE is determined as follows:
$$\% ( Blocked micro \text{-} organism rate ) = 100 - ( \frac{Na}{Nv} X 100 )$$
wherein:
Nv=concentration of the test micro-organism in the aerosol inside the test chamber (white test)
Na=concentration of the test micro-organism below the filtering face masks (sample test)

3. Methodology as claimed in claim 2 where a suspension of a known quantity of a micro-organism is fed up, through a peristaltic pump, into a nebuliser where the compressed air, passing through the nebulisation tube, creates an aerosol.
This aerosol is fed into a desiccation tube, where it is mixed with dry compressed air which comes separately from a flow line; the microbial aerosol droplets entering the desiccation tube, evaporate quickly and are moved to the test chamber with a constant flow.
The test chamber is set up of a hermetically sealed container having sealed walls, with gaskets, one of which can be opened to allow the operators to work.
The Sheffield head is put into the hermetic container and it is complete with connecting piping to the self-respirator and with piping for the withdrawal of air samples contained into the test chamber and of the air passing through the PPE.
The respirator machinery is made up of a pump and regulates the breathing frequency as per the typical human breathing.
The suction system sucks the dispersion of the micro-organisms into the test chamber to quantify them; the suction occurs through a vacuum pump that allows the withdrawal at a constant flow which is controlled through the flow regulators.
Both the white test and the sample test are carried out simultaneously through two separate lines, by bubbling the micro-organisms dispersions in an appropriate and pH controlled solution.
This dispersion for the white test is withdrawn from the test chamber through a first suction line and it bubbles in a first sterilised glass bubbler.
The dispersion for the sample test is withdrawn from the air passing through the PPE by means of a second suction line and it bubbles in a second sterilised glass bubbler.
At the end of the test, the bubblers are disconnected, the solutions are delivered into sterile containers and the count of the micro-organisms in the solutions is performed.

4. Methodology as claimed in claim 2 characterized in that the test chamber has Lexan walls, one of the them having a opening/closing folding system; the Sheffield head is complete with an accessory having three concentric tubes, two of which are linked to the self respirator and the third one, linked to the suction system, collects the air passed through the PPE at the mouth-nose level of the head; a fourth tube, linked to the suction system, is placed at the right eye level and withdraws the air contained into the test chamber; the respirator is made up of a piston pump, of valves and of an inverter which regulates the inhalation/exhalation speed.

5. Methodology as claimed in claim 2 characterized in that the respiratory frequency is ranged between 20 and 40 cycles/minute, the air volume is ranged between 1.5 and 3.5 litres per cycle and the suction system is turned on few minutes after the aerosol generator turning on, in order to allow the test chamber to fill up homogeneously.

6. Apparatus for the evaluation of the protection against biological agents of the personal protective equipment for the respiratory tract characterized in a viral/bacterial aerosol generator, a test chamber containing a Sheffield's head, a respirator simulating breathing and adjusting the inspiration and expiration frequency, a suction system which delivers the samples of air withdrawn in different points to the bubblers to determine the viral and/or bacterial concentrations; the Sheffield head being equipped with pipes connected to the self-respirator to allow the inhalation and the exhalation of the air through the mouth-nose area and with drawal pipes of both the air contained into the test chamber and the air passing through the PPE.

7. Apparatus as claimed in claim 6 characterized in that the aerosol generator includes a pump, a nebulisation line, a nebuliser, a desiccation tube and a flow line; the test chamber is set up of a hermetically sealed container with the Sheffield head complete with connecting piping to the self-respirator and with piping for the withdrawal of air samples contained into the test chamber and of the air passing through the PPE;
the respirator machinery is made up of a pump and regulates the inhalation/exhalation speed; the suction system is made up of a vacuum pump, flow regulators, a suction line with bubbler for the white test, a suction line with bubbler for the sample test.

8. Apparatus as claimed in claim 6 where the Sheffield head is placed into a test chamber in lexan, is complete with an accessory having three concentric tubes, two of which are linked to the self respirator and the third one collects the air passed through the PPE at the mouth-nose level of the head; a fourth tube is placed at the right eye level and withdraws the air contained into the test chamber; the respirator is made up of a piston pump, of valves and of an inverter which regulates the inhalation/exhalation speed.

9. Use of the Sheffield head for the evaluation of the protection of the personal protective equipments for the respiratory tract against biological agents.

10. Use of the Sheffield head, modified as described in claim 8, for the evaluation of the protection of the personal protective equipments for the respiratory tract against biological agents.

11. Use of a piston pump complete with an inverter to simulate breathing in the evaluation of the protection of the personal protective equipments for the respiratory tract against biological agents.

12. Methodology as claimed in claim 1 for the evaluation of the protection of the personal protective equipments for the respiratory tract against viral agents.

13. Methodology as claimed in claim 1 for the evaluation of the protection of the personal protective equipments for the respiratory tract against bacterial agents.

14. Methodology as claimed in claim 3 where the preparation of the micro-organisms suspensions is carried out by any process known for this use.

15. Methodology as claimed in claim 2 where the count of the micro-organisms is carried out by any process known for this use.
